(19) United States
(12) Patent Application Publication
(10) Pub. No.: US 2024/0173066 A1
(43) Pub. Date: May 30, 2024

(54) ORGAN HARVESTING TOOL

(71) Applicant: Maquet Cardiovascular LLC, Wayne, NJ (US)

(72) Inventors: Juan Isaias Perez, San Jose, CA (US); Ashik A. Mohan, Alamo, CA (US); Samuel R. Phillips, San Mateo, CA (US); Marc S. Kreidler, Sunnyvale, CA (US)

(73) Assignee: Maquet Cardiovascular LLC, Wayne, NJ (US)

(21) Appl. No.: 18/476,277

(22) Filed: Feb. 15, 2024

Related U.S. Application Data
(60) Provisional application No. 63/410,943, filed on Sep. 28, 2022.

(51) Int. Cl. A61B 18/14; A61B 17/00; A61B 17/28; A61B 17/29; A61B 18/00; A61B 90/00

(52) U.S. Cl. CPC A61B 18/1445; A61B 18/1492; A61B 2017/00969; A61B 2017/2825; A61B 2017/2905; A61B 2017/2913; A61B 2018/00083; A61B 2018/00595; A61B 2018/00601; A61B 2018/0063; A61B 2018/00946; A61B 2090/3983

(57) ABSTRACT

A surgical instrument for harvesting an organ includes a surgical tool and a handle. The handle includes an actuator rotatable about an actuator pivot pin and a cam connected to the actuator and rotatable in tandem with the actuator. The surgical instrument further includes an actuator rod having a distal end connected to the surgical tool and a proximal end connected to the cam. The cam defines a slot which captures a portion of the actuator rod. The portion of the actuator rod captured in the slot is slidable within the slot as the cam rotates. Sliding of the portion of the actuator rod in the slot actuates the surgical tool.

### CROSS-REFERENCE TO RELATED APPLICATIONS

This application claims the benefit of priority to U.S. Provisional Patent Application No. 63/410,943, filed Sep. 28, 2022, the contents of which are incorporated herein by reference in its entirety.

### FIELD OF THE DISCLOSURE

The present disclosure relates to a surgical instrument, and more particularly, to an organ harvesting device.

### DESCRIPTION OF RELATED ART

In endoscopic vessel harvesting (EVH) surgical procedures, a long slender surgical instrument may be advanced into a tunnel next to the saphenous vein in a patient's leg, and along the saphenous vein to dissect the vessel away from adjacent tissue, and to sever side-branch vessels along the course of the vessel to be harvested. Similar techniques may also be used to harvest a radial artery or other target structure.

Vessel harvesting devices often include a surgical tool at the distal end of the harvesting device and a handle with a control for operating the surgical tool. The handle is typically held in an operator's hand and the control is actuated by flexion of the operator's thumb or trigger finger. Harvesting procedures often require repeated flexion of the thumb or finger, which can lead to operator fatigue. For example, the control may need to be actuated between 20 and 60 times, at a force of between 6 and 7 pounds per actuation, for a single vessel harvesting procedure. To compensate for fatigue, the operator may adopt a hand position that results in inefficient use of the surgical tool and potential damage to both the harvesting device and the patient's tissue/organs.

Symmetrical control configurations often make using related art devices unintuitive. For example, such controls may be moveable in a proximal direction to activate an electrode at or in the surgical tool, and may be moveable in a distal direction to deactivate the electrode. If the control is symmetric with respect to the directions of operation, then an operator may become confused as to whether he/she is activating or deactivating the electrode.

### SUMMARY OF THE DISCLOSURE

In view of the foregoing, there exists a need for devices and methods for devices which are intuitive to operate and less fatiguing for the operator.

Embodiments of the present disclosure are directed to a surgical instrument for harvesting an organ, including a surgical tool and a handle. The handle includes an actuator rotatable about an actuator pivot pin, and a cam connected to the actuator and rotatable in tandem with the actuator. The surgical instrument further includes an actuator rod having a distal end connected to the surgical tool and a proximal end connected to the cam. The cam defines a slot which captures a portion of the actuator rod. The portion of the actuator rod captured in the slot is slidable within the slot as the cam rotates. Sliding of the portion of the actuator rod in the slot actuates the surgical tool.

In some embodiments, the slot defines a proximal pocket and a distal pocket. Moving a button of the actuator in a proximal direction causes the portion of the actuator captured in the slot to slide toward the proximal pocket. Moving the button of the actuator in a distal direction causes the portion of the actuator captured in the slot to slide toward the distal pocket.

In some embodiments, the surgical tool includes a primary jaw and a secondary jaw. Sliding of the portion of the actuator rod in the slot opens or closes the primary jaw and the secondary jaw relative to one another.

In some embodiments, the handle further includes one or more biasing elements configured to bias the actuator toward a null position in which the portion of the actuator rod captured in the slot is positioned between the proximal pocket and the distal pocket of the slot.

In some embodiments, the one or more biasing elements includes one or more springs disposed in a guide channel of the handle.

In some embodiments, the surgical tool includes a conductive element. The handle further includes a switch for supplying electrical current to the conductive element from a power source, and a switch linkage connected to the actuator or the cam via a pivot pin and configured to actuate the switch. In the null position of the actuator, the switch linkage does not actuate the switch. Rotation of the actuator about the actuator pivot pin greater than a predetermined distance beyond the null position causes the switch linkage to actuate the switch.

In some embodiments, the surgical tool includes a primary jaw and a secondary jaw. In the null position of the actuator, the primary jaw and the secondary jaw are closed relative to one another.

In some embodiments, the one or more biasing elements include a first compression spring and a second compression spring configured to bias the switch linkage toward an equilibrium position in which a force exerted by the first compression spring is balanced by a force exerted by the second compression spring. The equilibrium position corresponds to the null position of the actuator.

In some embodiments, the slot in the cam is non-linear.

In some embodiments, the actuator rod is flexible.

Other embodiments of the present disclosure are directed to a surgical instrument for harvesting an organ. The surgical instrument includes a handle including an actuator, and a surgical tool including a primary jaw and a secondary jaw configured to open and close relative to one another in response to movement of the actuator. The primary jaw includes a support structure, a conductive element having a distal tang, and a retaining insert disposed at a distal end of the support structure and defining a cavity for receiving the distal tang of the conductive element.

In some embodiments, the primary jaw further includes an insulating material applied around the support structure and between the support structure and the conductive element.

In some embodiments, the distal tang of the conductive element is bent towards the support structure.

In some embodiments, the handle further includes a switch for supplying electrical current to the conductive element from a power source.

In some embodiments, the retaining insert is made from an electrically nonconductive material and isolates the distal tang from the support structure.

In some embodiments, the primary jaw further includes a raised marker to indicate the position of the surgical tool to an operator.

In some embodiments, the raised marker extends at least partially around an exterior surface of the primary jaw.

In some embodiments, the raised marker has a substantially semicircular cross section.

In some embodiments, the raised marker in integrally formed with an insulating material of the primary jaw.

In some embodiments, the secondary jaw includes a raised marker to indicate the position of the surgical tool to an operator.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a perspective view of a surgical instrument in accordance with an embodiment of the present disclosure;

FIG. 2 is a partial side view of the surgical instrument of FIG. 1;

FIG. 3 is a side view of the surgical instrument of FIG. 1;

FIG. 4 is a perspective view of a surgical tool of the surgical instrument of FIG. 1, shown in an open position;

FIG. 5 is a perspective view of the surgical tool of FIG. 4, shown in a closed position;

FIG. 6 is a perspective view of the surgical tool of FIG. 4, with an insulating cover removed for clarity;

FIG. 7 is a top view of the primary jaw of the surgical tool of FIG. 6;

FIG. 8 is a perspective cross-sectional view of the surgical tool of FIG. 5;

FIG. 9 is a cross-sectional view of the surgical tool of FIG. 5 cauterizing a vessel during a surgical procedure;

FIG. 10 is a perspective view of the surgical tool of FIG. 4 with insulating layers shown transparently for clarity;

FIG. 11 is a side perspective view of the surgical tool of FIG. 4 with insulating layers shown transparently for clarity;

FIG. 12 is a partial exploded view of the surgical tool of FIG. 1;

FIG. 13 is a side cutaway view of a handle of the surgical instrument of FIG. 1;

FIG. 14 is an electrical schematic of a switch and associated components of the surgical instrument of FIG. 1;

FIG. 15 is an electrical schematic of the switch and cable of FIG. 14;

FIG. 16 is a perspective view of the actuator, internal circuitry, and associated components of the surgical instrument of FIG. 1;

FIG. 17 is a left side view of the actuator of the surgical instrument of FIG. 1; and

FIG. 18 is a right side view of the actuator of FIG. 17.

### DETAILED DESCRIPTION OF THE DISCLOSURE

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a component of a surgical instrument, the term “proximal” refers to a portion of said component farthest from a surgical access site of the patient. When used in relation to a component of a surgical instrument, the term “distal” refers to a portion of said component nearest to, or inserted in, the patient.

As used herein, the term “surgical tool” refers to any device or component that may be used to operate on tissue (e.g., to treat, manipulate, handle, hold, cut, heat, or energize, etc., tissue).

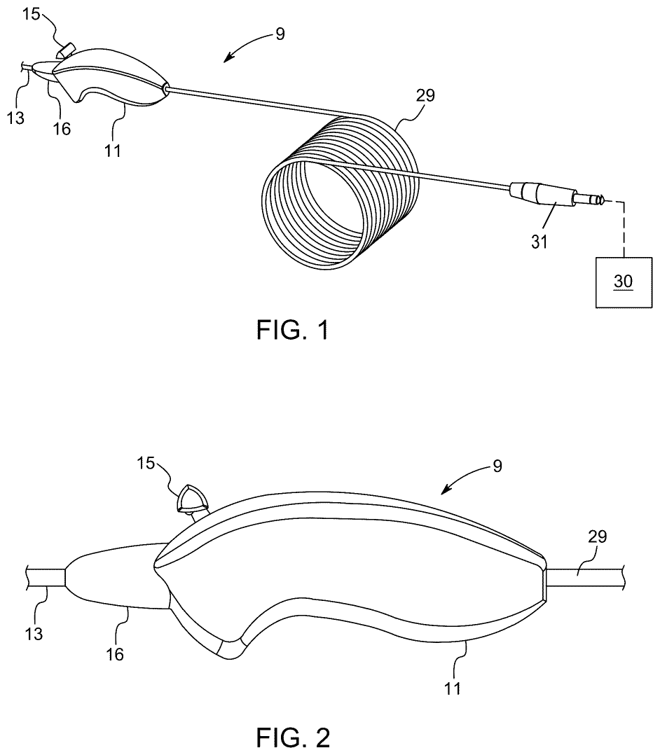

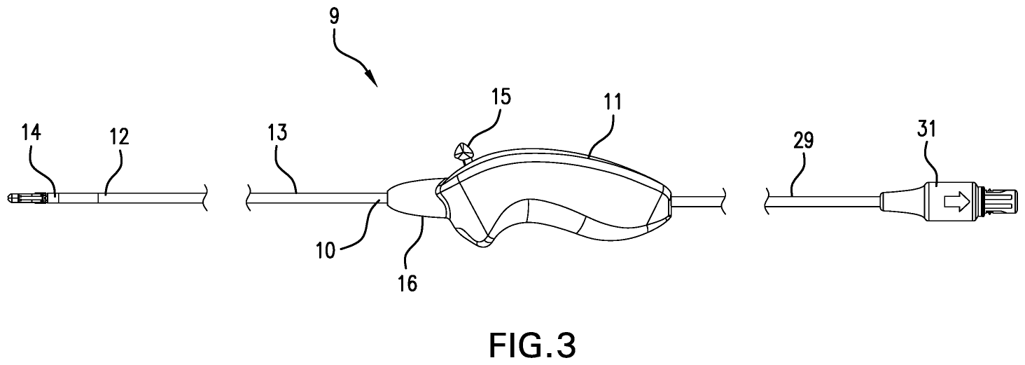

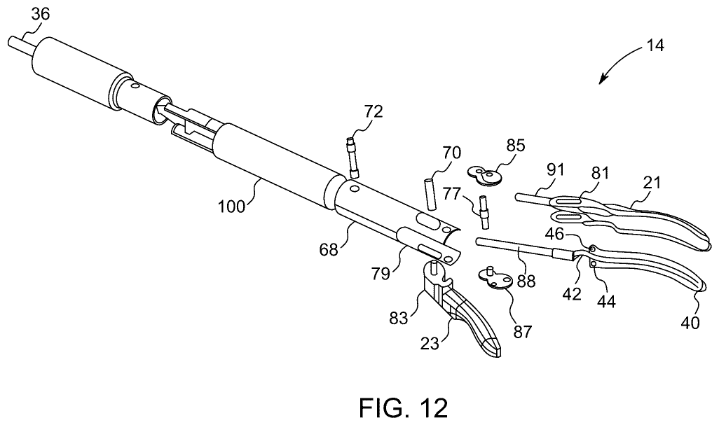

Referring now to the drawings in which like reference numerals refer to like parts, embodiments of the present disclosure are directed to a surgical instrument, particularly for use in endoscopic vessel harvesting (EVH) surgical procedures. Referring first to FIGS. 1 to 3, a surgical instrument 9 in accordance with embodiments of the present disclosure includes a handle 11, an elongated body 13 having a proximal end 10 and a distal end 12, and a surgical tool 14 located at the distal end 12 of the elongated body 13. The proximal end 10 of the elongated body 13 is coupled to a distal end 16 of the handle 11. The elongated body 13 may be rigid or, alternatively, flexible. The handle 11 includes an actuator 15 that is coupled to the surgical tool 14 through an actuator rod 36 (see FIG. 12) within a bore of the elongated body 13 for controlling an operation of the surgical tool 14. The actuator rod 36 may be one or more cables, shafts, gears, or any one of other suitable mechanical devices. The handle 11 and the actuator 15 may be made from insulating materials such as plastic.

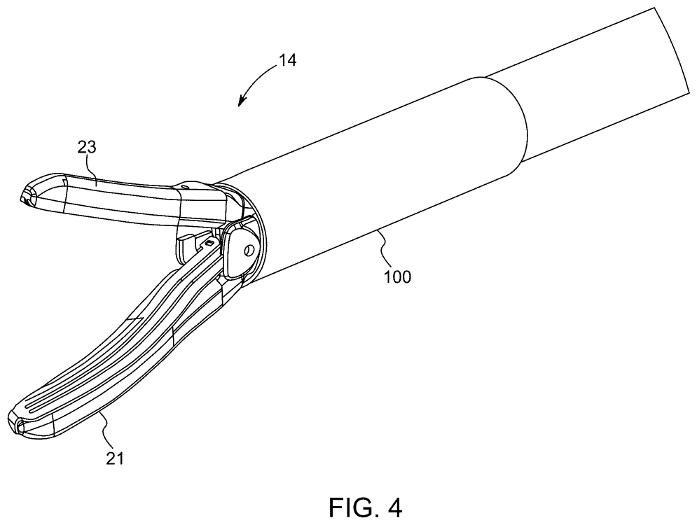

With continued reference to FIGS. 1 to 3 and further reference to FIGS. 4 to 9, the surgical tool 14 includes a pair of opposing jaws, namely a primary jaw 21 and a secondary jaw 23, for clamping, cutting, and sealing a vessel. The primary jaw 21 includes an electrically conductive element 40 which faces towards the secondary jaw 23. Alternatively, or additionally, the secondary jaw 23 may include an electrically conductive element which faces towards the primary jaw 21. The conductive element 40 may be in a form of an electrode, and is configured to selectively transfer heat during use. As used in this specification, the term “electrode” refers to a component that is for delivering energy, such as heat energy, and should not be limited to a component that delivers any particular form of energy. The electrically conductive element 40 may be made from nickel-chrome, stainless steel, or other metals or alloys in different embodiments. The jaws 21, 23 are configured to close in response to actuation (e.g., pressing, pulling, or pushing, etc.) of the actuator 15, thereby clamping a vessel during performance of a surgical procedure. The actuator 15 may be a two-stage actuator such that actuation (e.g., pressing, pulling, or pushing, etc.) initially causes the jaws 21, 23 to close, and further actuation (e.g., further pressed, further pulled, or further pushed, etc.) causes the conductive element 40 to provide (e.g., emit) heat, thereby cutting and sealing the clamped vessel. In particular, when the actuator 15 is further actuated, the conductive element 40 is electrically coupled, via a cable 29, to a direct current (DC) power source 30 (scc FIG. 1) which provides a current to the conductive element 40 and thereby heats the conductive element 40. After the vessel is cut and sealed, the actuator 15 may be de-actuated to stop the delivery of current to the conductive element 40, and may be further de-actuated to open the jaws 21, 23. In other embodiments, the source 30 may be other types of energy source, and need not be a DC source. The cable 29 may include a plug-type connector 31 that facilitates simple, user-friendly connection of the surgical instrument 9 to the source 30. In some embodiments, the cable 29 may include three conductor wires, and the connector 31 may include three pins corresponding to the three conductor wires of the cable 29.

With continued reference to FIGS. 1 to 12, the actuator rod 36 that mechanically couples the jaws 21, 23 to the actuator 15 may be electrically insulated, for example, by silicone rubber, ceramic, plastic heat-shrink material, or other suitable non-electrically conductive material. This assures that energy is safely conducted along the electric line housed by the elongated body 13 to the conductive element 40 at the primary jaw 21 (and/or a conductive element of the secondary jaw 23). In other embodiments, the elongated body 13 may not include an electric line for coupling to the conductive element 40. Instead, the actuator rod 36 may be electrically conductive, and is used to transfer energy to the conductive element 40.

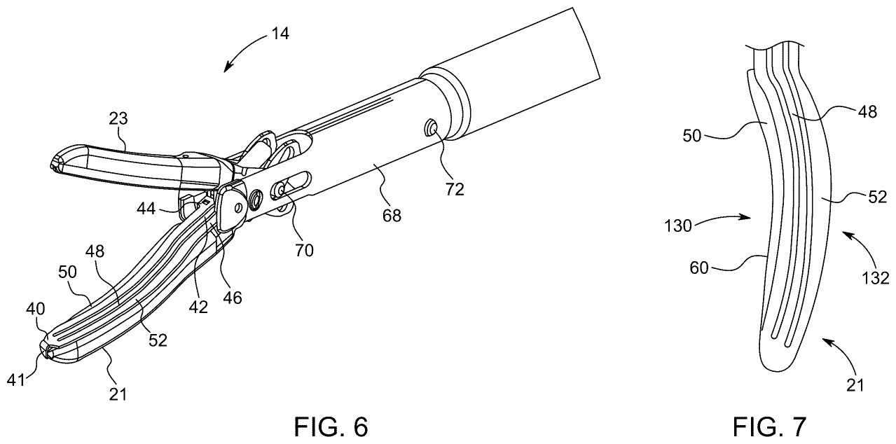

Referring now to FIGS. 6 to 12, the surgical tool 14 and, particularly, the pair of jaws 21, 23 are shown in greater detail. The conductive element 40 disposed on a surface of the primary jaw 21 includes two outer portions 50, 52, and an inner (middle) portion 48. The outer portions 50, 52 have respective outer terminals 44, 46 at their ends, and the middle portion 48 has an inner terminal 42 at its end. Thus, the outer and inner portions 48, 50, 52 form an electrical heater circuit between the inner terminal 42 and the outer terminals 44, 46. In the embodiments illustrated in the drawings, the outer portions 50, 52 and the inner portion 48 of the conductive element 40 function as an electrode that is configured to deliver heat to operate on a vessel. In particular, the terminal 42 of the electrode 40 is electrically coupled to a first terminal of the DC source 30 (sec FIG. 1), and outer terminals 44, 46 of the electrode 40 are electrically coupled to a second terminal of the DC source 30, thereby allowing the electrode 40 to receive and conduct DC energy for cutting and/or welding tissue. The conductive element 40 may be formed using a single, flat sheet of electrically conductive material (e.g., nickel-chrome alloy, such as stainless steel at an outer layer, and nickel-chrome at an inner layer). This construction has reliability, manufacturing, and cost advantages. It also reduces the likelihood of tissue buildup and entrapment during use by minimizing crevices into which tissue can migrate.

During use, current from the DC source 30 (see FIG. 1) is conducted through the inner terminal 42, and flows in the inner portion 48 of the conductive element 40 and in parallel through the dual outer portions 50, 52 of the conductive element 40 to the outer terminals 44, 46. Thus, for inner and outer portions 48, 50, 52 of equal thicknesses and equal widths, current density in the inner portion 48 is twice as high as the current density in each of the outer portions 50, 52 in response to an electrical signal (e.g., voltage) applied between inner terminal 42 and the outer terminals 44, 46. Of course, current densities in the inner and outer portions 48, 50, 52 may be altered (for example, by altering the relative widths of the inner and/or outer portions 48, 50, 52, by altering resistances through selection of different materials, by altering both the widths and resistances, etc.) to alter the operating temperatures of the inner and outer portions 48, 50, 52. In operation, the outer portions 50, 52 may operate at a temperature sufficient to weld a tissue structure (e.g., a blood vessel) grasped between the jaws 21, 23, and the inner portion 48 may operate at a higher temperature sufficient to sever the grasped tissue structure intermediate of the welded segments.

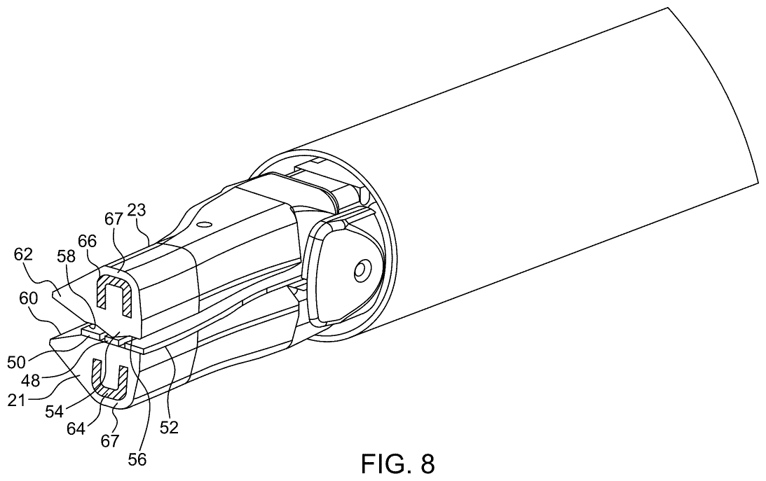

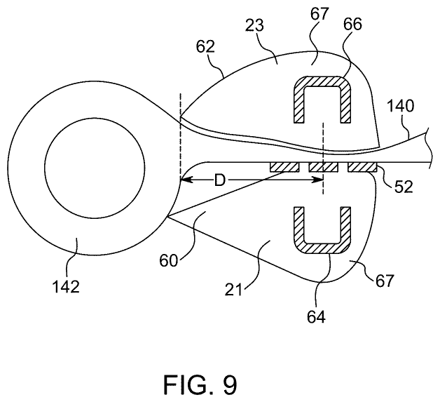

Referring now to FIGS. 8 and 9, shown is a partial cross sectional view of the jaws 21, 23 that illustrates the placement of inner and outer portions 48, 50, 52. The primary jaw 21 includes a structural support 64, and the secondary jaw 23 includes a structural support 66. In some embodiments, the structural supports 64, 66 may be made from electrically conductive material that allows the supports 64, 66 to function as electrical lines (e.g., for transmitting electrical current). For example, the structural supports 64, 66 may be made of stainless steel. The structural supports 64, 66 are covered by one or more layers of electrically insulating material 67, such as rubber, polymers, silicone, polycarbonate, ceramic or other suitable insulating material. The insulating material 67 may be molded separately and bonded onto the respective structural supports 64, 66. Alternatively, the insulating material 67 may be over-molded onto the structural supports 64, 66. For example, each of the structural supports 64, 66 may have one or more openings for allowing the insulating material 67 to flow therethrough during the over-molding process. A priming material, such as silicone primer, may be applied to the structural supports 64, 66 prior to application and/or molding of the insulating material 67 to improve adhesion of the insulating material 67 to the structural supports 64, 66. The priming material thus reduces the occurrence of detachment of the insulating material 67 and the conductive element 40 during use of the surgical instrument 9.

With continued reference to FIG. 8, the secondary jaw 23 includes a surface elevation or protrusion 54 substantially in alignment with the inner (middle) portion 48 of the primary jaw 21 in order to increase the compression force applied to a tissue structure grasped by the jaws 21, 23 and in contact with the middle portion 48. The protrusion 54 promotes more efficient tissue severance, while adjacent regions 56, 58 of the surface elevation 54 on the secondary jaw 23 in alignment with the outer portions 50, 52 of the conductive element 40 introduce less compression force suitable for welding grasped tissue.

Referring now to FIGS. 7 to 9, the cross sections of the respective jaws 21, 23 may not be symmetrical. Instead, the primary jaw 21 may have a protrusion 60, and the secondary jaw 23 may have a protrusion 62. Each of the protrusions 60, 62 extends generally perpendicular to the support structures 64, 66 of the respective jaws 21, 23 so as to appropriately space the surgical tool 14 from a main vessel 142 of the patient during a surgical procedure. In particular, during a procedure for removing a main vessel 142 by cutting a branch vessel 140, the protrusions 60, 62 abut the main vessel 142 such that the jaws 21, 23 are at a prescribed or predetermined distance D spaced apart from the main vessel 142, as shown in FIG. 9. For example, the predetermined distance D may be at least 1 mm, and more preferably, at least 1.5 mm. However, the predetermined distance D may be any value sufficient to prevent or minimize thermal spread from the conductive element 40 to the main vessel 142. As such, the protrusions 60, 62 help prevent or minimize thermal spread to the main vessel 142 from the cutting and sealing of the branch vessel 140, thereby preserving the integrity of the main vessel 142 that is being harvested. Also, the protrusions 60, 62 obviate the need for an operator to guess whether the cutting of the branch vessel 140 is sufficiently far (e.g., beyond a minimum prescribed spacing) from the main vessel 142. Instead, the operator merely abuts the protrusions 60, 62 of the surgical tool 14 against the main vessel 142, and the protrusions 60, 62 will automatically position the jaws 21, 23 relative to the branch vessel 140 so that the branch vessel 140 is cut at the predetermined distance D from the main vessel 142. In some cases, if the surgical instrument 9 is used to cut other types of tissue, such as nerves, organs, tendons, etc., the protrusions 60, 62 also provide the same benefits of preserving the integrity of tissue adjacent to the cut and obviating the need for an operator to guess the appropriate margin and/or position of the surgical tool 14.

As shown in FIG. 9, the protrusions 60, 62 may diverge or taper away from the branch vessel 140. Such a configuration allows part of the branch vessel 140 that is immediately next to the main vessel 142 not to be clamped by the jaws. As a result, the severed end of the branch vessel 140 will fall away once it is cut. In other embodiments, the surgical instrument 9 does not need to include both protrusions 60, 62. Instead, the surgical instrument 9 may include only one of the protrusion 60 or protrusion 62. Such a configuration allows the device at the distal end of the surgical instrument 9 to have a smaller profile, thereby allowing an operator to effectively maneuver the surgical tool 14 in tight tissue conditions. As shown in FIG. 9, the outer portion 52 may protrude laterally along an outer edge of the closed jaws 21, 23.

With continued reference to FIGS. 6 to 9, the primary jaw 21 may have a concave side 130 and a convex side 132. In one method of use, while the jaws 21, 23 are used to cut a branch vessel 140, the primary jaw 21 is oriented so that the concave side 130 faces towards the main vessel 142. An endoscope or viewing device may be placed in proximity to the jaws 21, 23 with the endoscope or viewing device viewing the concave side 130 of the primary jaw 21. This allows the operator to better visualize the tips of the jaws 21, 23. The configuration of the concave side 130 and the convex side 132 also provides a safety benefit by allowing the operator to know where the tips of the jaws 21, 23 are during the vessel cutting procedure. As shown in FIG. 9, a protruding section of the outer portion 52 is on the convex side 132 of the primary jaw 21, while the protrusions 60, 62 are on the concave side 130 of the primary jaw 21. The concavity of the concave side 130 provides extra spacing to further protect the main vessel 142 when the branch vessel 140 is grasped. Furthermore, the exposed outer portion 52 on the convex side 132 creates a protrusion that makes it easier to contact the wall of the tunnel in the patient's body with the protruding section of the outer portion 52 to address bleeding. In other embodiments, the protrusions 60, 62 may be on the convex side 132 of the jaw assembly while the protruding section of the outer portion 52 is on the concave side 130. In such cases, during use, the convex side 132 of the jaws 21, 23 would be oriented towards the main vessel 142, thereby ensuring that the tips of the jaws 21, 23 do not inadvertently contact the main vessel 142, thereby preventing the jaws 21, 23 from injuring the main vessel 142.

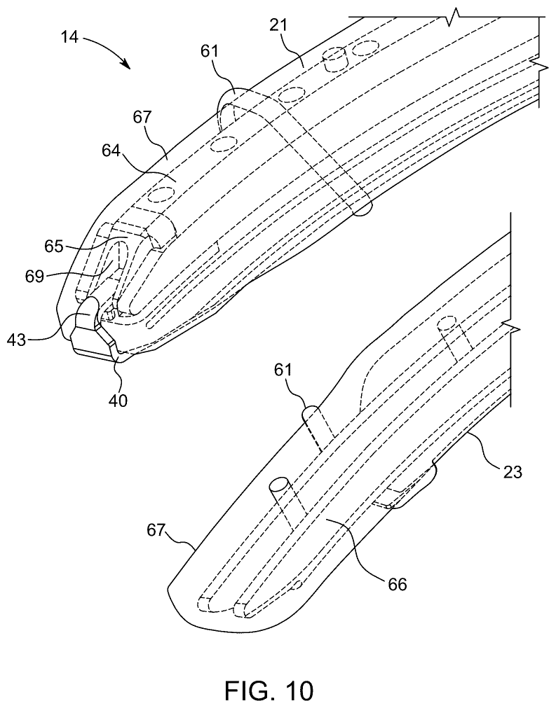

Referring now to FIG. 10, the primary jaw 21 may include a retaining insert 65 for securing a distal tang 43 of the conductive element 40. The retaining insert 65 may be disposed at a distal end of the structural support 64. The distal tang 43 of the conductive element 40 may be bent towards the structural support 64 so as to extend into the retaining insert 65. The retaining insert 65 may be a high-temperature resistant polymer applied to the structural support 64 prior to over-molding, or otherwise forming, the insulating material 67 onto the structural support 64. As such, the retaining insert 65 may be mechanically held in place between structural support 64 and the insulating material 67. The retaining insert 65 may define a cavity 69 that receives the distal tang 43 of the conductive element 40, thereby preventing the distal end 41 of the conductive element 40 from detaching from the primary jaw 21. The insulating material 67 may include an opening corresponding to the cavity 69 of the retaining insert 65 through which the distal tang 43 of the conductive element 40 may pass to enter the cavity 69. The retaining insert 65 may isolate the distal tang 43 of the conductive element 40 from the support structure 64, and may be made of an electrically nonconductive material so that the distal tang 43 does not create a short circuit with the support structure 64.

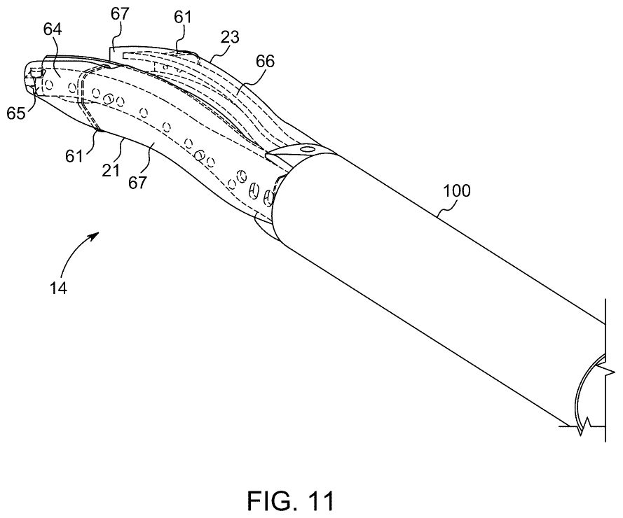

With continued reference to FIG. 10 and further reference to FIG. 11, the insulating material 67 of the primary jaw 21 may include a raised marker 61 that indicates the location and/or the position of the conductive element 40 to assist the operator in positioning the jaws 21, 23 for cutting. The insulating material 67 of the secondary jaw 23 may likewise include a raised marker 61 to assist the operator in positioning the jaws 21, 23 for cutting. The markers 61 on each of the jaws 21, 23 may be in the form of a ridge which protrudes radially from the respective jaw 21, 23. Each marker 61 may have a substantially semicircular cross section and may extend at least partially around an exterior surface of the jaw 21, 23. The markers 61 may alternatively have other cross-sectional profiles such as rectangular, triangular, rounded, polygonal, etc. The markers 61 may be formed integrally with the insulating material 67, for example during an over-molding process in which the insulating material 67 is applied to the jaws 21, 23. The markers 61 may be reflective to improve visibility to the operator.

Referring now to FIG. 12, components of a jaw-operating mechanism of the surgical tool 14 may be supported in a rod housing 68 that includes sliding pin 70 and attachment pin 72, all covered with an insulating cover 100. The insulating cover 100 may be made of a flexible material such as silicone rubber, plastic heat-shrink material, or the like, to shield/protect adjacent tissue from moving parts of the surgical tool 14 and from electrical energy within the surgical instrument 9. The insulating cover 100 may also retain the sliding pin 70 and the attachment pin 72 in place to obviate the need for more complex fasteners and mechanisms.

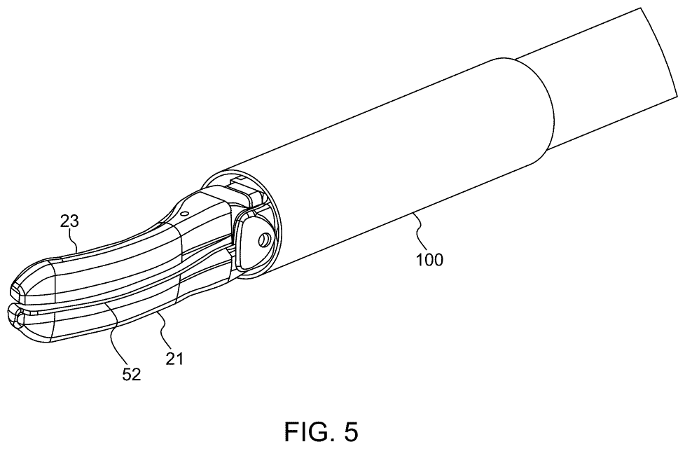

With continued reference to FIG. 12, there is illustrated an exploded view of the components forming the surgical tool 14, and attachment to the distal end of the elongated body 13. Specifically, the conductive element 40, including the inner and outer portions 48, 50, 52, is attached to the primary jaw 21. Both the primary jaw 21 and the secondary jaw 23 are pivotally attached via insulating material clevises 85, 87 and a jaw pin 77 to a rod housing 68. The jaws 21, 23 pivot on the clevises 85, 87 so that the jaws 21, 23 can be kept electrically isolated from the jaw pin 77 which holds the inner terminal 42 of the conductive element 40 against the face of the primary jaw 21. This configuration prevents the structural supports 64, 66 (which may be metal) of jaws 21, 23 from contacting the jaw pin 77, thereby avoiding an electrical short circuit. A slide pin 70 is disposed to slide within aligned slots 79 in the housing 68, and within the mating angled slots 81, 83 in the primary jaw 21 and the secondary jaw 23, respectively. Movement of the slide pin 70 relative to the jaw pin 77 effects a scissor-like motion of the jaws 21, 23 between an open position (as shown, for example, in FIGS. 4 and 6) and a closed position (as shown, for example, in FIG. 5). The actuating rod 36 is linked to the slide pin 70, for example, via a yoke that is attached to the distal end of the actuator rod 36. Axial movement of the actuating rod 36 in one direction will cause the slide pin 70 to move towards the jaw pin 77, thereby opening the jaws 21, 23. Axial movement of the actuating rod 36 in the opposite direction will cause the slide pin 70 to move away from the jaw pin 77, thereby closing the jaws 21, 23.

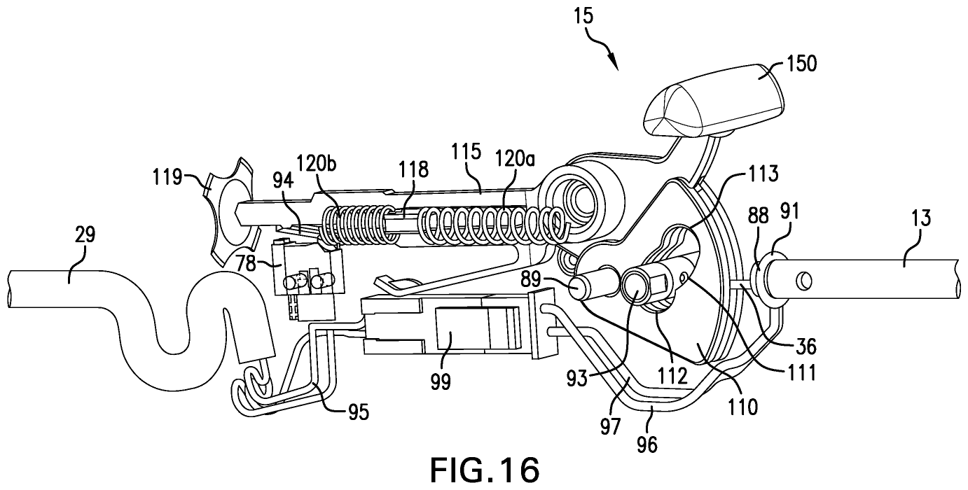

With continued reference to FIG. 12, an electrical conductor 88 connects to the inner terminal 42 of the conductive element 40, and the outer terminals 44, 46 are electrically connected in common to an electrical conductor 91. The electrical conductor 88 or the electrical conductor 91 extend through the elongated body 13 such that the electrical conductors 88, 91 can be accessed from a proximal end of the elongate body 13 as shown in FIG. 16. In other embodiments, if the actuating rod 36 is electrically conductive, the electrical conductor 88 and/or the electrical conductor 91 may be coupled to the actuating rod 36. In such embodiment, the actuating rod 36 will be electrically coupled to one terminal of the DC source 30, or to the contact 95 of the switch 78 (see FIG. 16), during use. During use, the electrical conductors 88, 91 may be electrically coupled to terminals of the DC source 30, which provides a current to thereby heat the inner and outer portions 48, 50, 52 of the conductive element 40. The center inner portion 48 is configured to cut a vessel (e.g., a branch vessel 140) while the outer portions 50, 52 are configured to weld (seal) the vessel. In some embodiments, parts of the surgical tool 14 may be insulated via an insulating cover 100 for isolating certain components from biologic tissue and fluids.

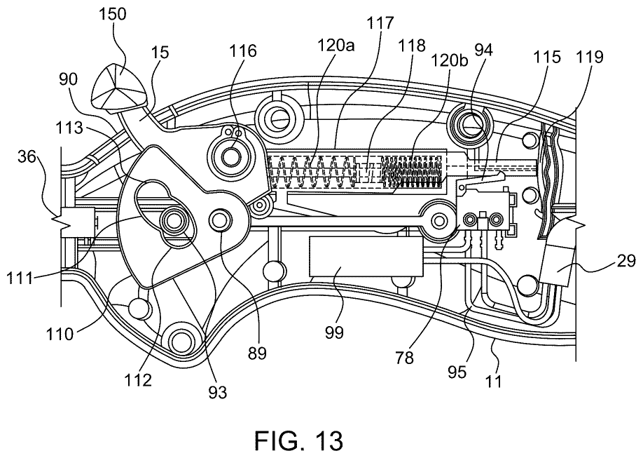

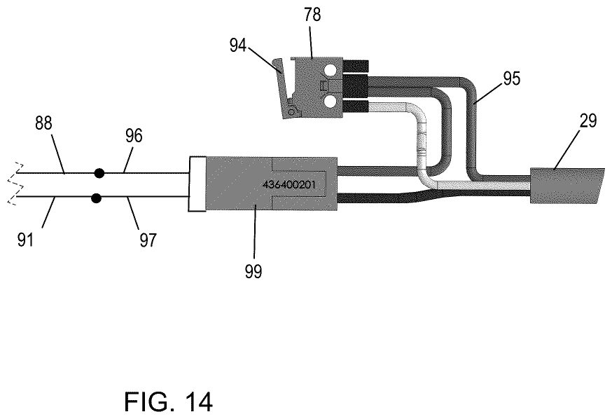

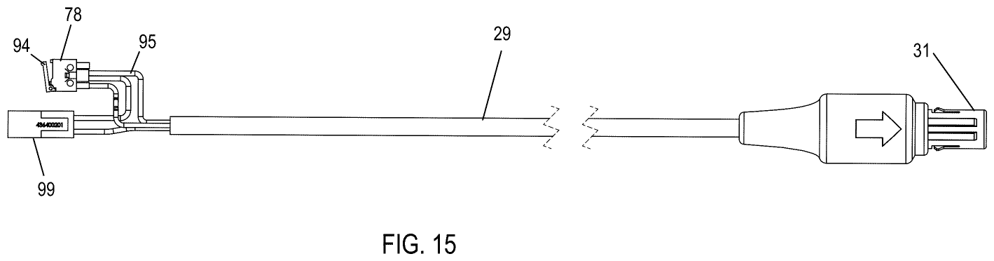

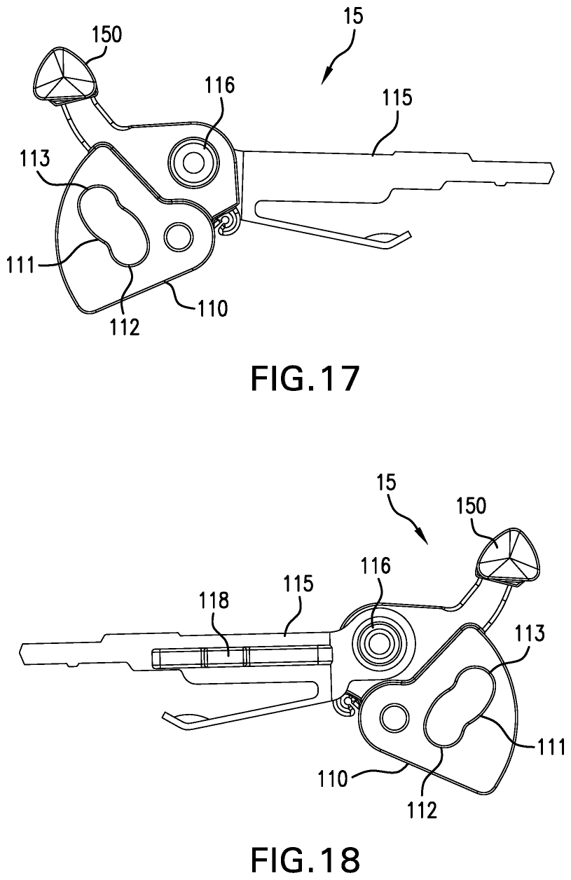

Referring now to FIGS. 13 to 18, the internal components of the handle 11 are shown. The handle 11 may be formed of two half-sections to facilitate assembly of the surgical instrument 9. FIG. 13 shows the components installed in one half section of the handle 11. A complementary half section (not shown) that snaps together with, or is otherwise attached to, the illustrated half section to enclose the components within the handle 11. The handle 11 may be formed of plastic material that provides electrical insulation, ergonomics, and durability. In some cases, the material for construction of the handle 11 is selected so that it provides adequate strength for the handle 11 to withstand forces of the mechanisms and forces of the operator interacting with the instrument 9 during a procedure.

An electrical switch 78 is mounted in the handle 11 to be operated at least partially in conjunction with the actuator 15 for controlling electrical power supplied to the inner and outer portions 48, 50, 52 of the conductive element 40. The actuator 15 is rotatably mounted to the handle 11 via an actuator pivot pin 89 so that the actuator 15 is pivotable relative to the handle 11. Referring now to FIGS. 13 to 16, the switch 78 may be a normally open switch which, when engaged by a portion of the actuator 15, closes a circuit to supply electrical power from the DC source 30 (see FIG. 1) via the cable 29 to the conductive elements 40. In particular, the cable 29 may be electrically connected to a connector 99 disposed in the handle 11, with at least one conductor 95 of the cable 29 interrupted by the switch 78. The connector 99 is attached to conductor wires 96, 97 supplying electrical power to the electrical conductors 88, 91 connected to the conductive element 40. In an unengaged position, as shown in FIG. 13, a button 150 of the actuator 15 is in a null position and the switch 78 is disengaged. As such, the circuit controlled by the switch 78 is open and no power is supplied to the electrical conductors 88, 91 connected to the conductive element 40. Moving the button 150 proximally closes the switch 78, as will be described in greater detail below, thereby supplying power from the cable 29 to the conductive element 40.

With continued reference to FIG. 13, the actuator 15 extends through a slot 90 in the handle 11. The actuator 15 can rotate about the actuator pivot pin 89 when the operator applies a proximal or distal force to a button 150 of the actuator 15. Within the handle 11, a cam 110 is attached to the actuator 15 and rotates in tandem with the actuator 15 about the actuator pivot pin 89. The cam 110 defines a slot 111 which captures a portion of the actuating rod 36, e.g. barrel 93 of the actuating rod 36, thereby mechanically linking the actuating rod 36 to the actuator 15. The barrel 93 is slidable within the slot 111 as the cam 110 is rotated about the actuator pivot pin 89. The slot 111 of the cam 110 defines a proximal pocket 112 and a distal pocket 113. In the null position of the actuator 15, as shown in FIG. 13, the barrel 93 is positioned between the proximal pocket 112 and the distal pocket 113 of the slot 111 so that the button 150 can be moved either distally or proximally. If the button 150 of the actuator 15 is pushed distally by the operator, rotation of the actuator 15 and the cam 110 guides the barrel 93 of the actuating rod 36 into the distal pocket 113 of the slot 111 causing the actuating rod 36 to translate in a distal direction. Distal translation of the actuating rod 36 opens the jaws 21, 23, as described herein in connection with FIG. 12. Similarly, if the button 150 of the actuator 15 is pulled proximally by the operator, rotation of the actuator 15 and the cam 110 guides the barrel 93 of the actuating rod 36 into the proximal pocket 112 of the slot 111 causing the actuating rod 36 to translate in a proximal direction. Proximal translation of the actuating rod 36 closes the jaws 21, 23, as described in connection with FIG. 12.

With continued reference to FIGS. 13 to 16, the actuator 15 may be mechanically coupled to the switch 78 in order to control electrical power supplied to the inner and outer portions 48, 50, 52 of the conductive element 40, as previously indicated. In particular, a switch linkage 115 may be connected to the actuator 15 and/or the cam 110 by a pivot pin 116. When the button 150 of the actuator 15 is pulled in the proximal direction to close the jaws 21, 23, the switch linkage 115 is pulled proximally by the actuator 15. In so doing, a proximal end of the switch linkage 115 engages a contact pad 119 formed in the handle 11. The contact pad 119 directs the switch linkage 115 toward a lever 94 of the switch 78. Continued proximal pulling of the button 150 of the actuator 15 causes the switch linkage 115 to depress the lever 94 of the switch 78. As the lever 94 is depressed by switch linkage 115, an electrical circuit is completed and electrical current is provided to the conductive element 40 via the switch 78. Conversely, when the button 150 of the actuator 15 is pushed in the distal direction to open the jaws 21, 23, the switch linkage 115 is pulled distally by the actuator 15 and releases the lever 94 of the switch 78 to interrupt electrical current to the conductive element 40. As described herein in connection with FIGS. 1 to 3, the actuator may have a two-stage operation, in which initial proximal pulling of the actuator 15 less than a predetermined distance (or degree of rotation) beyond the null position retains the jaws 21, 23 in a closed position relative to one another, but does not supply electrical power to the heating element 40. To produce the two-stage operation, the geometry of the actuator 15, the cam 110, and the switch linkage 115 is such that the actuator 15 can be pulled proximally a sufficient distance to close the jaws 21, 23 without the switch linkage 115 depressing the lever 94 of the switch 78. If the actuator 15 is then pulled further than the predetermined distance in the proximal direction, the jaws 21, 23 remain closed while the switch linkage 115 moves further in the proximal direction to depress the lever 94 and supply current to the conductive element 40.

With continued reference to FIGS. 13 and 16, the actuator 15 may be biased towards a null position such that, in the absence of a force applied to the button 150, the actuator 15 automatically returns to the null position. In the null position, the switch 78 is open so that no electrical power is supplied to the conductive element 40, and the jaws 21, 23 are closed to allow easy navigation of the surgical tool 14 through the surgical access site of the patient. Because the actuator 15 is automatically biased toward the null position, the operator does not have to exert any energy to keep the jaws 21, 23 closed and the conductive element 40 unpowered during positioning of the surgical tool 12 in the patient. To bias the actuator 15 toward the null position, the handle 11 may include one or more springs or other biasing elements. In the embodiment shown in FIGS. 13 and 16, the handle 11 defines a spring guide channel 117 which houses a pair of opposing compression springs 120a. 120b. The compression springs 120a. 120b engage opposing faces of a spring tab 118 of the switch linkage 115, thereby biasing the spring tab 118 to an equilibrium position in which the force exerted by a first of the compression springs 120a is balanced by a force exerted by a second of the compression springs 120b. The equilibrium position of the spring tab 118 corresponds to the null position of the actuator 15 such that as the compression springs 120a. 120b move the spring tab 118 of the switch linkage 115 to the equilibrium position, the switch linkage 115 in turn moves the actuator 15 to the null position.

With reference to FIGS. 13, 16, 17, and 18, the geometry of the cam 110 may be selected to reduce the force required for the operator to move and maintain the jaws 21, 23 in the open position and/or closed position. In some embodiments, the cam 110 may produce an over-center action which assists the operator in moving the actuator 15 and in maintaining the actuator 15 in either direction of travel from the null position. In particular, the slot 111 may be non-linear, such as an S-shape, such that as the button 150 is pulled proximally beyond the null position, the barrel 93 of the actuator rod 36 is induced toward the proximal pocket 112 of the slot 111, thereby reducing the force required to be exerted on the button 150. Similarly, as the button 150 is pushed distally beyond the null position, the barrel 93 of the actuator rod 36 is induced toward the distal pocket 113 of the slot 111, thereby reducing the force required to be exerted on the button 150. In some embodiments, the geometry of the actuator 15 and the cam 110 may reduce the force needed to open or close the jaws 21, 23 to approximately 2 pounds.

Referring to FIGS. 1 to 18, when the actuator 15 is pushed distally to open the jaws 21, 23, the opened jaws 21, 23 can be used to surround a target tissue (e.g., the branch vessel 140 as shown in FIG. 9). When the jaws 21, 23 are placed around target tissue, the actuator 15 may be pulled proximally to close the jaws 21, 23 thereby gripping the target tissue. If desired, the actuator 15 may be further pulled proximally such that the switch linkage 115 depresses the lever 94 of the switch 78, thereby supplying DC power from the DC source 30 to the conductive element 40. As previously described herein, the switch 78 does not close, and therefore does not supply DC power, until the actuator 15 is moved proximally by a predetermined distance beyond the null position. As such, DC power for cutting and/or cauterizing the target tissue is not supplied until after the jaws 21, 23 have gripped the tissue, and the operator has further pulled the actuator 15. This prevents the conductive element 40 of the jaws 21, 23 from being prematurely powered. Delivery of DC power may be stopped by pushing the actuator 15 distally (or simply by releasing the button 150 so that the actuator 15 returns to the null position under the influence of compression springs 120a. 120b) such that the switch linkage 115 disengages the lever 94 of the switch 78.

During use of the surgical instrument 9, the elongated body 13 is advanced along a vessel, e.g. the main vessel 142 shown in FIG. 9, to be harvested. In some cases, the surgical instrument 9 may be placed into an instrument channel of a cannula which includes a viewing device, such as an endoscope, for allowing an operator to see the distal end of the surgical instrument 9 inside the patient. Examples of suitable cannulas with which the surgical instrument 9 may be used are describe in U.S. provisional patent application entitled “Cannula for Use With an Endoscopic Vessel Harvesting Device”, attorney docket no. CS.917, filed on Sep. 28, 2022, in the name of Maquet Cardiovascular LLC, the disclosure of which is hereby incorporated by reference in its entirety. When the branch vessel 140 (or other target tissue) is encountered, the jaws 21, 23 may be used to grasp and compress the branch vessel 140 in response to manipulation of the actuator 15. Power is then supplied using the DC source 30 to the inner and outer portions 48, 50, 52 of the conductive element 40 (which function as resistive elements that heat up in response to the delivered direct current) to effect tissue welds at tissues that are in contact with outer portions 50, 52, and to effect tissue cutting at tissue that is in contact with inner portion 48.

During the vessel harvesting procedure, if the operator notices that there is bleeding in the surrounding tissues (e.g., from the walls of the surgical cavity), the operator may utilize the conductive element 40 to cauterize the bleeding tissue. The conductive element 40 serves as a DC electrode to electrocauterize any tissue (e.g., vessel tissue or surrounding tissue) that is grasped between the jaws 21, 23. Alternatively, the protruding section of the outer portion 52 of the conductive element 40 that protrudes from the side of the primary jaw 21 (as shown in FIG. 9) may be used to cauterize a bleeding area. In such cases, the jaws 21, 23 may or may not be closed, and may or may not be grasping any tissue. For example, in some embodiments, the operator may not be using the jaws 21, 23 to grasp or cut tissue. However, if the operator notices that there is bleeding at or near the surgical site, the operator may use the protruding section of the outer portion 52 of the conductive element 40 to cauterize the bleeding area. In particular, the protruding section of outer portion 52 serves as a DC electrode for electrocauterizing the tissue. For example, the side or the tip of the outer portion 52 that extends beyond the profile of the primary jaw 21 may be used to perform thermal spot cauterization by direct thermal conduction. In such cases, the outer portion 52 may be heated up, and the protruding section may be used to touch tissue that is desired to be cauterized.

As illustrated in the above embodiments, the surgical instrument 9 allows delivery of heat to a remote surgical site for welding and severing vessel. Embodiments of the surgical instrument 9 also obviate the need for repeatedly inserting a separate bleeding control device inside the patient to control bleeding, and removing such bleeding control device from the patient, during a vessel harvesting procedure. Thus, embodiments of the surgical instrument 9 described herein more easily and efficiently addresses bleeding.

Although the above embodiments have been described with reference to the surgical tool 14 being a pair of jaws for clamping, cutting, and sealing vessel (e.g., saphenous vein, an artery, or any other vessel), in other embodiments, the surgical tool 14 may have different configurations, and different functionalities. For example, in other embodiments, the surgical tool 14 may be clip appliers or grasping jaws. In further embodiments, the bleeding control feature may be incorporated in any type of laparoscopic/endoscopic surgical tool, or any type of tool used for open surgery. Also, in any of the embodiments described herein, the surgical instrument 9 may be used in any endoscopic procedure that requires dissection or transection of tissue with bleeding control.

Also, although the above embodiments have been described with reference to a surgical instrument that has a bleeding control feature, in other embodiments, such bleeding control feature is optional. In addition, in any of the embodiments described herein, the surgical tool 14 at the distal end of the surgical instrument 9 does not need to include all of the features described herein. For example, in some embodiments, the surgical tool 14 does not include the outer portions 50, 52 of the conductive element 40. Instead, the surgical tool 14 may include one electrode strip (comparable to the middle electrode portion 48 of the conductive element 40 described herein) for cutting or sealing tissue. Furthermore, in other embodiments, the secondary jaw 23 may not have the surface elevation 54. Instead, the secondary jaw 23 may have a flat surface for contacting the inner and outer portions 48, 50, 52 of the conductive element 40. In addition, in further embodiments, the jaws 21, 23 may not include the respective protrusions 60, 62. Instead, the cross section of the jaw 21/23 may have a symmetrical configuration. In other embodiments, protrusions may be provided on both sides of the jaw assembly (e.g., one or more protrusions at the concave side 130 of the jaws 21, 23, and one or more protrusions at the convex side 132 of the jaws 21, 23). Such configuration provides buffering on both sides of the surgical tool 14, and allows for correct placement of the surgical tool 14 regardless of which side (the concave side 130 or the convex side 132) of the surgical tool 14 is oriented towards the main vessel 142 during use. In further embodiments, instead of the curved configuration, the jaws 21, 23 could be straight. Also, in any of the embodiments described herein, instead of, or in addition to, using the conductive element 40 for controlling bleeding, the conductive element 40 may be used for dissection or transection of tissue, such as fatty and connective tissue encountered during a vessel harvesting procedure.

While examples of organ harvesting devices were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A surgical instrument for harvesting an organ, comprising:
a surgical tool;
a handle comprising:
an actuator rotatable about an actuator pivot pin; and
a cam connected to the actuator and rotatable in tandem with the actuator;
an actuator rod having a distal end connected to the surgical tool and a proximal end connected to the cam,
wherein the cam defines a slot which captures a portion of the actuator rod,
wherein the portion of the actuator rod captured in the slot is slidable within the slot as the cam rotates, and
wherein sliding of the portion of the actuator rod in the slot actuates the surgical tool.

2. The surgical instrument of claim 1, wherein the slot defines a proximal pocket and a distal pocket,
wherein moving a button of the actuator in a proximal direction causes the portion of the actuator captured in the slot to slide toward the proximal pocket, and
wherein moving the button of the actuator in a distal direction causes the portion of the actuator captured in the slot to slide toward the distal pocket.

3. The surgical instrument of claim 1, wherein the surgical tool comprises:
a primary jaw; and
a secondary jaw,
wherein sliding of the portion of the actuator rod in the slot opens or closes the primary jaw and the secondary jaw relative to one another.

4. The surgical instrument of claim 2, wherein the handle further comprises one or more biasing elements configured to bias the actuator toward a null position in which the portion of the actuator rod captured in the slot is positioned between the proximal pocket and the distal pocket of the slot.

5. The surgical instrument of claim 4 wherein the one or more biasing elements comprises one or more springs disposed in a guide channel of the handle.

6. The surgical instrument of claim 4, wherein the surgical tool comprises a conductive element,
wherein the handle further comprises:
a switch for supplying electrical current to the conductive element from a power source; and
a switch linkage connected to the actuator or the cam via a pivot pin and configured to actuate the switch,
wherein in the null position of the actuator, the switch linkage does not actuate the switch, and
wherein rotation of the actuator about the actuator pivot pin greater than a predetermined distance beyond the null position causes the switch linkage to actuate the switch.

7. The surgical instrument of claim 6, wherein the surgical tool comprises:
a primary jaw; and
a secondary jaw,
wherein, in the null position of the actuator, the primary jaw and the secondary jaw are closed relative to one another.

8. The surgical instrument of claim 6, wherein the one or more biasing elements comprise a first compression spring and a second compression spring configured to bias the switch linkage toward an equilibrium position in which a force exerted by the first compression spring is balanced by a force exerted by the second compression spring, and
wherein the equilibrium position corresponds to the null position of the actuator.

9. The surgical instrument of claim 1, wherein the slot in the cam is non-linear.

10. The surgical instrument of claim 1, wherein the actuator rod is flexible.

11. A surgical instrument for harvesting an organ, comprising:
a handle comprising an actuator;
a surgical tool comprising a primary jaw and a secondary jaw configured to open and close relative to one another in response to movement of the actuator,
wherein the primary jaw comprises:
a support structure;
a conductive element having a distal tang; and
a retaining insert disposed at a distal end of the support structure and defining a cavity for receiving the distal tang of the conductive element.

12. The surgical instrument of claim 11, wherein the primary jaw further comprises an insulating material applied around the support structure and between the support structure and the conductive element.

13. The surgical instrument of claim 11, wherein the distal tang of the conductive element is bent towards the support structure.

14. The surgical instrument of claim 11, wherein the handle further comprises a switch for supplying electrical current to the conductive element from a power source.

15. The surgical instrument of claim 11, wherein the retaining insert is made from an electrically nonconductive material and isolates the distal tang from the support structure.

16. The surgical instrument of claim 11, wherein the primary jaw further comprises a raised marker to indicate the position of the surgical tool to an operator.

17. The surgical instrument of claim 16, wherein the raised marker extends at least partially around an exterior surface of the primary jaw.

18. The surgical instrument of claim 16, wherein the raised marker has a substantially semicircular cross section.

19. The surgical instrument of claim 16, wherein the raised marker in integrally formed with an insulating material of the primary jaw.

20. The surgical instrument of claim 11, wherein the secondary jaw comprises a raised marker to indicate the position of the surgical tool to an operator.
